(19) United States
(12) Patent Application Publication
(10) Pub. No.: US 2017/0056190 A1
(43) Pub. Date: Mar. 2, 2017

(54) SUBTALAR BIOFOAM WEDGE

(71) Applicant: Wright Medical Technology, Inc., Memphis, TN (US)

(72) Inventors: Jennifer GUILFORD, Memphis, TN (US); Joseph Ryan WOODARD, Memphis, TN (US); Chris ROBINSON, Hernando, MS (US)

(21) Appl. No.: 14/837,695

(22) Filed: Aug. 27, 2015

(51) Int. Cl. A61F 2/42; A61F 2/46; A61F 2/30

(52) U.S. Cl. CPC A61F 2/4202; A61F 2/30767; A61F 2/4684; A61F 2002/4207; A61F 2002/3093; A61F 2002/30932; A61F 2310/00023; A61F 2002/30131; A61F 2002/30126; A61F 2002/4217

(57) ABSTRACT

A subtalar implant includes a body having a sidewall defining an outer perimeter of the body. The sidewall defines an inner volume. A porous material is disposed within the inner volume. The porous material has a porosity configured to promote bone ingrowth. The porosity of the porous material can be about 30% to about 70% by volume. The sidewall can include a smooth surface configured to prevent bone ingrowth.

### FIELD OF DISCLOSURE

This disclosure generally relates to orthopedic medical implant devices for surgical joint fusion. More particularly, the disclosed subject matter generally relates to a joint fusion implant for the bones of the human foot, especially the subtalar joint.

### BACKGROUND

Orthopedic implant devices have been utilized to fully or partially replace existing skeletal joints in humans. There are many joints in the human foot, such as the subtalar joint, which frequently suffer from abnormal wear or other defects.

A subtalar fusion is a common surgical procedure for correction of calcaneal fractures, abnormal wear of the subtalar joint, flatfoot deformity, and/or other abnormalities in the subtalar joint. Fusion of the subtalar joint is generally achieved with calcaneal screws. Current solutions do not correct angular deformities that may be present in the subtalar joint, for example, in patients with flatfoot deformities.

### SUMMARY

In various embodiments, a subtalar implant is disclosed. The subtalar implant includes a body having a sidewall defining an outer perimeter of the body. The sidewall defines an inner volume. A porous material is disposed within the inner volume. The porous material has a porosity configured to promote bone ingrowth. The porosity of the porous material can be about 30% to about 70% by volume. The sidewall can be a smooth, solid structure configured to prevent bone in-growth.

In some embodiments, a subtalar implant system is disclosed. The subtalar implant system includes an implant and a bone screw. The implant includes a body having a solid sidewall defining an outer perimeter of the body. The solid sidewall defines an inner volume. The implant further includes a porous metal material disposed within the inner volume, the porous metal material having a porosity of about 30% to about 70% by volume. The bone screw is sized and configured for fusing a subtalar joint.

In some embodiments, a method of correcting a subtalar joint deformity is disclosed. The method includes preparing a subtalar joint for receiving an implant. The implant includes a body having a sidewall defining an outer perimeter of the body. The sidewall defines an inner volume. A porous material is disposed within the inner volume. The porous material has a porosity configured to promote bone ingrowth. The implant is positioned in the prepared subtalar joint. A screw is driven through a first bone of the subtalar joint into a second bone of the subtalar joint to fuse the first and second bones.

### BRIEF DESCRIPTION OF THE FIGURES

The features and advantages of the present invention will be more fully disclosed in, or rendered obvious by the following detailed description of the preferred embodiments, which are to be considered together with the accompanying drawings wherein like numbers refer to like parts and further wherein:

FIG. 1A illustrates one embodiment of an orthopedic implant having a solid sidewall and a porous internal material in accordance with the present disclosure.

FIG. 1B illustrates a side view of the orthopedic implant of FIG. 1A.

FIG. 2 illustrates one embodiment of an orthopedic implant coupled between a first bone and a second bone of a subtalar joint in accordance with the present disclosure.

FIG. 3A illustrates one embodiment of a midfoot wedge implant in accordance with the present disclosure.

FIG. 3B illustrates a side view of the midfoot wedge implant of FIG. 3A.

FIG. 4A illustrates one embodiment of an orthopedic implant having a full-oval shape in accordance with the present disclosure.

FIG. 4B illustrates a side view of the orthopedic implant of FIG. 4A

FIG. 5 illustrates one embodiment of an insertion tool configured to insert an orthopedic implant to a surgical site in accordance with the present disclosure.

FIGS. 6A-6E illustrates one embodiment of a trial for the implant system in accordance with the present disclosure.

FIG. 7 is a flowchart illustrating one embodiment of a method for inserting an orthopedic implant at a joint in accordance with the present disclosure.

FIGS. 8A-8E illustrates one embodiment of a surgical technique for installing an implant in accordance with the present disclosure.

FIGS. 9A-9C illustrates one embodiment of a surgical technique for lateral installation of an implant in accordance with the present disclosure.

FIG. 10 is a flowchart illustrating one embodiment of a method of manufacturing an orthopedic implant in accordance with the present disclosure.

### DETAILED DESCRIPTION

This description of the exemplary embodiments is intended to be read in connection with the accompanying drawings, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected,” refer to a relationship wherein structures are secured or attached to one another either directly or indirectly through intervening structures, as well as both movable or rigid attachments or relationships, unless expressly described otherwise.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

For brevity, “orthopedic implant devices,” “orthopedic implant,” “implant” and the like are used interchangeably in the present disclosure. References to “orthopedic implants,” or “implants” made in the present disclosure will be understood to encompass any suitable device configured to fuse, fix or partially replace a joint between two bones, including but not limited to a subtalar implant.

References to “solid” or “substantially solid” are made relative to references to “porous” and “substantially porous.” Unless expressly indicated otherwise, references to “solid” or “substantially solid” made below will be understood to describe a material or structure having 0-5% by volume (e.g., 0-2% by volume) of porosity. A small amount of pores, particularly closed pores, may be embedded inside a solid or substantially solid material.

Unless expressly indicated otherwise, references to “porous” or” substantially porous” made below will be understood to describe a material or structure having a significant amount of pores, for example, higher than 5% by volume of porosity. A porous or substantially porous materials may have pores, particularly open pores on the surface. The porosity on or adjacent to the surface may be higher than 5% by volume in some embodiments. When a material monolith is porous, the porosity may be in the range from 20-95% (e.g., 50-80%) by volume.

Data of pore size and porosity were measured following the FDA's guidance: “Guidance Document for Testing Orthopedic Implants With Modified Metallic Surfaces Apposing Bone or Bone Cements,” 1994. Each part was sectioned using electric discharge machining to produce smooth and even surfaces that represent cross-sections through the porous material. Green modeling clay was used to fill the pores of the cut face. A razor blade was used to remove any excess modeling clay from the cross section. Images were taken at 75× magnification using a Zeiss microscope with a camera attachment. Parts were oriented in a way to give best possible color contrast between the titanium and the modeling clay. Simagis image analysis software (Smart Imaging Technology, Houston, Tex.) was used to determine the percent porosity, strut diameter, interconnecting pore diameter and pore cell diameter. The pore size (or interconnecting pore size) was defined as the approximately circular pore opening that connects larger pore cells.

In various embodiments, the present disclosure generally provides an orthopedic implant for use in a joint, such as a subtalar joint, during bone fusion. The implant comprises a sidewall defining a predetermined shape having an inner volume. The inner volume is filled with a porous material. The sidewall defines at least one opening configured to expose a portion of the porous material. The porous material has a predetermined porosity to facilitate bone ingrowth where desired. The sidewall is configured to support at least a portion of a load experienced by the joint.

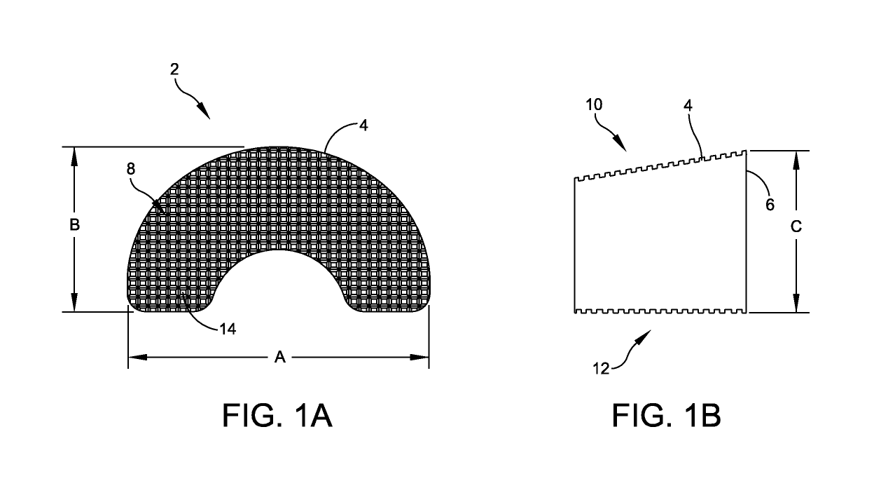

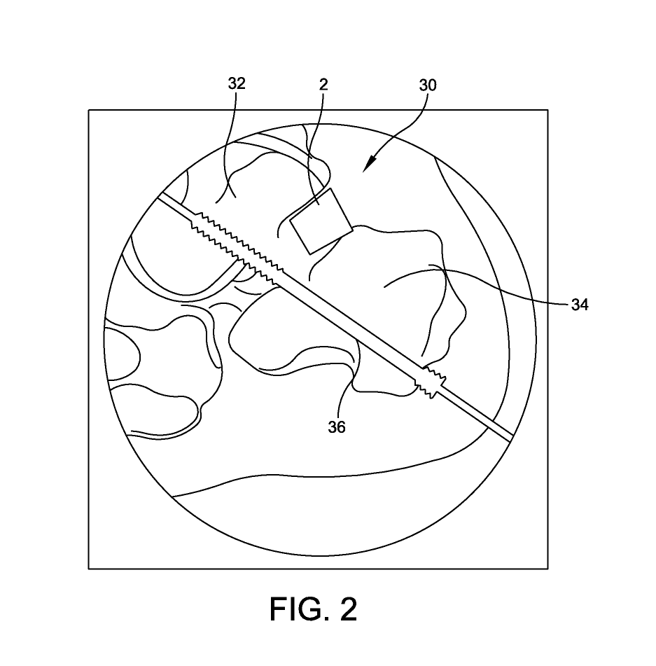

FIGS. 1A and 1B illustrate one embodiment of an orthopedic implant 2. The implant 2 comprises a body 4 having a sidewall 6. The sidewall 6 has a predetermined shape defining an inner volume 8. For example, in some embodiments, the sidewall 6 defines a horse-shoe shape, a crescent shape, a cylindrical shape, a partial-oval shape, a full-oval shape, and/or any other suitable shape. In the illustrated embodiment, the sidewall 6 defines a horse-shoe shape. In some embodiments, the body 4 may be a unitary body. The sidewall 6 may also be referred to as an “external surface,” “smooth surface,” or “an outer surface” of the implant 2.

In some embodiments, the sidewall 6 defines a perimeter of the body 4 having at least one opening, such as, for example, an open top edge 10 and/or an open bottom edge 12. For example, in the illustrated embodiment, the sidewall 6 of the body 4 defines a horse-shoe shape having an open top edge 10 and an open bottom edge 12. In some embodiments, the sidewall 6 can partially and/or completely cover the top edge 10 and/or the bottom edge 12 of the body 4. In other embodiments, a portion of the sidewall 6 may be omitted to expose a section of the internal volume 8 along the perimeter of the sidewall 6.

In some embodiments, the sidewall 6 comprises a solid material, such as, for example, a solid metal. For example, in some embodiments, the sidewall 6 comprises a metal having a porosity of less than about 5% by volume. In some embodiments, the sidewall 6 comprises a substantially smooth surface. The material of the sidewall 6 may be selected to inhibit soft tissue ingrowth onto and/or through the sidewall 6. Suitable exemplary biocompatible materials include, but are not limited to, titanium, titanium-alloys, steel, and/or alloy steel.

In some embodiments, the internal volume 8 defined by the sidewall 6 is filled with a porous material 14. The internal volume 8 may be partially and/or completely filled with the porous material 14. The porous material 14 may have pores of any suitable size or ranges. For example, The pore size may be in the range of from about 1 micron to about 2000 microns in diameter, for example, from about 100 microns to about 1000 microns in diameter, or in the range of from about 400 microns to about 600 microns in diameter. The pores can be continuous and open. The porosity can be in the range from about 30% to about 70% by volume in some embodiments, such as, for example, from about 50% to 70%, from about 55% to about 65%, and/or any other suitable range. The porous material 14 may comprise any suitable biocompatible material.

In some embodiments, the porous material 14 is made of porous titanium such as, for example, BIOFOAM® available from Wright Medical Inc., although other porous materials can be used. BIOFOAM® is made of commercially pure titanium and has pores, for example, of roughly 500 microns in diameter. The porosity can be up to 70% by volume. Such porous titanium has continuous and open pores. The porous titanium or titanium alloy mimics the strength and flexibility of human bone, and also has a high surface coefficient of friction.

In some embodiments, the porous material 14 has at least one exposed surface having a predetermined porosity and is configured to promote bone fixation through friction and bone ingrowth. In various embodiments, pore size may be in the range of from about 1 micron to about 2000 microns in diameter, for example, from about 100 microns to about 1000 microns in diameter, or in the range of from about 400 microns to about 600 microns in diameter. In some embodiments the porous material 14 has exposed surfaces at the top edge 10 and/or bottom edge 12 of the sidewall 6. The exposed surfaces of the porous material 14 are positioned to interact with an implantation site to promote bone ingrowth through the internal volume 8. In some embodiments, a bone-growth agent is included within the porous material 14 to encourage bone ingrowth.

In some embodiments, the implant 2 is configured to support a predetermined load. The predetermined load can correspond to a load experienced by a joint and/or a bone into which the implant 2 is inserted. For example, in some embodiments, the implant 2 is a subtalar implant configured to support a maximum force experienced by a subtalar joint of a patient. In other embodiments, the implant 2 is configured to support some multiple of the force experienced by the joint and/or the bone, such as, for example, 1.5 times the maximum force, twice the maximum force, three times the maximum force, and/or any other suitable multiple. The solid sidewall 6 and the porous material 14 allow the implant 2 to support loads greater than the strength of the porous material 14 alone while providing the flexibility and bone ingrowth of a porous material 14. The porous material 14 is configured to contact bone at the implantation site to promote bone ingrowth and increase the strength of the implant/bone connection. The sidewall 6 prevents compression and/or distortion of the porous structure 14 when a force greater than the compression force of the porous material 14 is experienced at the implantation site.

In some embodiments, the implant 2 is sized and configured for implantation at a joint, such as, for example, a subtalar joint. The implant 2 may be configured to correct one or more defects of the subtalar joint, such as, for example, a flatfoot deformity. However, one of ordinary skill in the art will understand that implant 2 can be used to fuse, fix, and/or partially replace another joint between two bones.

The implant 2 can be of any suitable size, which can be determined by the size of the joint and associated bones. Table 1 lists some exemplary embodiments of implants for subtalar joints.

TABLE 1
Exemplary Subtalar Implants of Different Sizes

| | | Width of Implant | Height of Implant |
|---|---|---|---|
| | Length of Implant | (Dimension B) | (Dimension C) |
| Example | (Dimension A) (mm) | (mm) | (mm) |
| 1 | 25 | 14.5 | 15 |
| 2 | 25 | 14.5 | 20 |
| 3 | 25 | 14.5 | 25 |

In some embodiments, one or more of the dimensions of the implant 2 may be variable. As shown in FIG. 1B, in some embodiments, the height C of the implant 2 may vary from a proximal portion of the implant to a distal portion of the implant. For example, in some embodiments, the proximal portion of the implant 2 may have a height C and the distal portion of the implant may have a height less than C. The top edge 10 of the sidewall 6 tapers from the proximal end of the implant 2 to the distal end of the implant 2. In other embodiments, one or more of the length A, width B, height C, and/or any other dimension may be variable.

FIG. 2 illustrates one embodiment of the implant 2 configured for, and implanted at, a subtalar joint 30. The subtalar joint 30 consists of a joint between the talus 32 and the calcaneus 34 of the foot. Prior to insertion of the implant 2, the talus 32 and/or the calcaneus 34 is resected to remove a portion of the bone 32, 34 to accommodate the implant 2. Although an implant 2 configured to the subtalar joint 30 is discussed herein, it will be appreciated that the implant 2 can be used to fuse, fix, and/or partially replace another joint between two bones and is not limited to joints of the foot.

The implant 2 is located within the subtalar joint 30 to correct angular deformities of the subtalar joint 30, such as a flatfoot deformity. The sidewall 6 of the implant 2 provides for angular correction of the subtalar joint 30 while providing the mechanical strength necessary to hold full ankle loading. In some embodiments, the implant 2 is paired with a bone screw 36 configured to fuse the subtalar joint 30. In some embodiments, the body of the implant 2 includes a shape configured to allow implantation of the bone screw 36 according to one or more known implantation techniques. For example, in some embodiments, the implant 2 has a horse-shoe shape sized and configured to allow for implantation of the bone screw 36 according to one or more known methods.

In some embodiments, the implant 2 is positioned in the joint 30 such that at least one open side 10, 12 of the implant 2 is in contact with the talus 32 and/or the calcaneus 36. The open side(s) 10, 12 allows a porous material 14 located within a cavity 8 to contact the surface of bones 32, 34 to promote bone ingrowth. As discussed above, the porous material 14 has a predetermined porosity and surface roughness parameter configured to promote bone ingrowth. For example, in some embodiments, the porous material 14 includes a BIOFOAM® material having a porosity of up to 70% by volume.

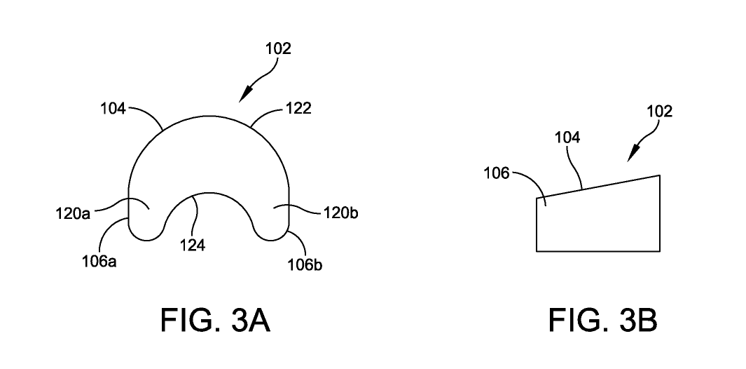

FIGS. 3A and 3B illustrate one embodiment of a midfoot wedge implant 102 having elongated end portions 120a, 120b. The implant 102 is similar to the implant 2 described in conjunction with FIGS. 1-3, and similar description is not repeated herein. In some embodiments, the implant 102 includes a sidewall 106 having an outer curve 122 and an inner curve 124. The outer curve 122 extends from a first elongated end portion 120a to a second elongated end portion 120b along an outside perimeter of the implant 102. The inner curve 124 extends from the first elongated end portion 120a to the second elongated end portion 120b along an inside perimeter of the implant 102. The elongated end portions 120a, 120b comprise ends of the implant 102 having flat sidewalls 106a, 106b. The flat sidewalls 106a, 106b extend the distal ends portions 120a, 120b of the implant 102 beyond the curvature of the outer curve 122 and the inner curve 124. The implant 102 is configured for insertion at one or more joints, such as, for example, a midfoot joint.

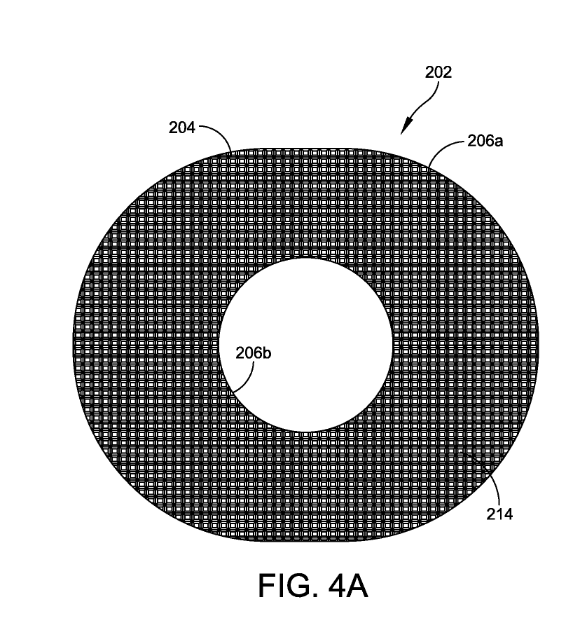

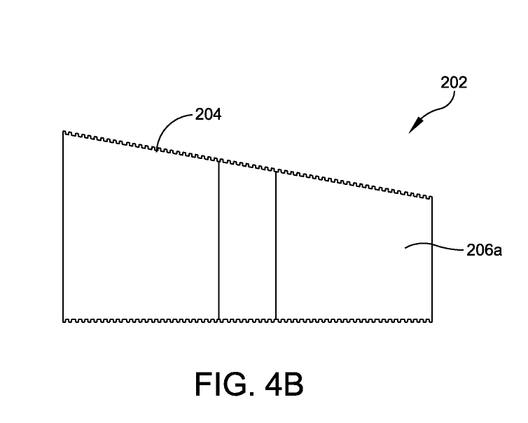

FIGS. 4A and 4B illustrate one embodiment of an implant 202 having a full-oval (or “race-track”) cross section. The implant 202 is similar to the implant 2 described in conjunction with FIGS. 1-3, and similar description is not repeated herein. In some embodiments, the implant 202 comprises an outer wall 206a and an inner wall 206b. The outer wall 206a and the inner wall 206b define respective concentric oval shapes. The outer wall 206a has a first diameter and the inner wall 206b has a second, smaller diameter. A porous material 214 is disposed between the outer wall 206a and the inner wall 206b. The porous material 214 has a predetermined porosity configured to promote bone ingrowth. For example, in some embodiments, the porous material 214 has a porosity of between about 30% to about 70%. In some embodiments, the porous material includes a BIOFOAM® material. The outer wall 206a and/or the inner wall 206b of the implant 202 may comprise a smooth surface to prevent soft tissue ingrowth, allowing bone ingrowth only through the porous material 214. For example, in some embodiments, the outer wall 206a and/or the inner wall 206b include a solid titanium wall.

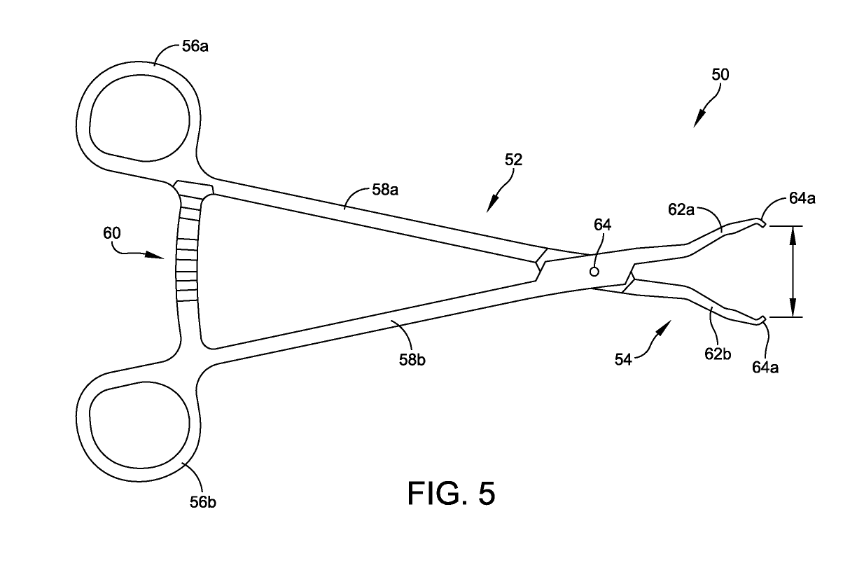

FIG. 5 illustrates one embodiment of an insertion tool 50 configured to insert an orthopedic implant 2 to a prepared joint. The insertion tool 50 includes a proximal handle portion 52 and a distal head portion 54. The handle portion 52 includes a first finger ring 56a and a second finger ring 56b. A first longitudinal shaft 58a extends distally from the first finger ring 56a and a second longitudinal shaft 58b extends distally from the second finger ring 56b. In some embodiments, the head portion 54 comprises a first gripping portion 62a and a second gripping portion 62b pivotally coupled at pivot point 64. The gripping portions 62a, 62b may be integrally formed with the first and second longitudinal shafts 58a, 58b. In some embodiments, the gripping portions 62a, 62b include a curved distal end 66a, 66b configured to partially wrap around an implant located within the head portion 54. The insertion tool 50 is operated in a scissor-like manner to hold and/or release an implant during insertion. In some embodiments, the insertion tool 50 includes a locking mechanism 60 for locking the head portion 54 of the insertion tool 50 at a predetermined rotation.

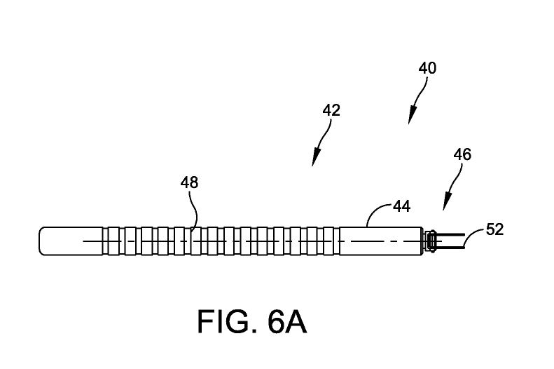

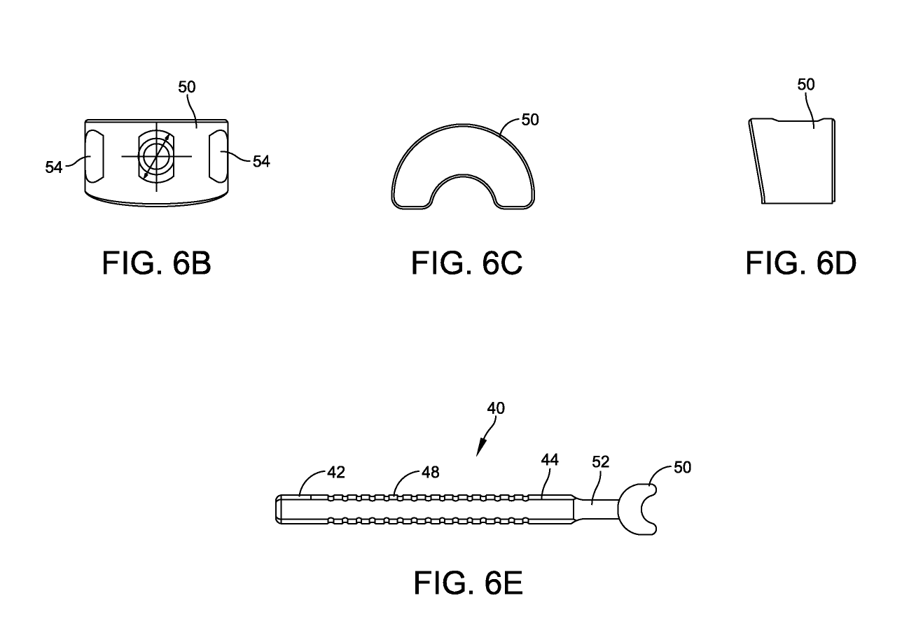

FIGS. 6A-6E illustrates one embodiment of a trial system 40, such as, for example, a midfoot trial. The trial 40 comprises a handle 42 having an elongate shaft 44. A distal end 46 of the shaft is sized and configured to releasably couple to a trial 50 (see FIG. 6B). In some embodiments, the shaft 42 comprises a plurality of gripping features 48 formed thereon. The gripping features 48 may comprise, for example, a plurality of channels formed in the elongate shaft 42 and spaced along the length of the elongate shaft 42. The gripping features 48 are configured to increase control of the elongate shaft 42 during positioning of a trial 50. The trial system 40 is sized and configured for insertion into a resected joint to determine a implant size prior to insertion of the implant. In some embodiments, the handle 40 comprises one or more protrusions 52 for coupling the handle 42 to the trial 50. For example, in the illustrated embodiment, the distal end 46 of the handle 42 includes first and second protrusions 52. The protrusions 52 are sized and configured to be received within cavities 54 formed in the trial 50. The size of the cavities 54 can be consistent over multiple sized trials 50 to ensure proper fit between the protrusions 52 and the cavities 54.

During surgery, the trial system 40 is used to determine an appropriately sized implant for insertion into a resected joint. After the joint has been prepared by the surgeon, the surgeon selects a trial 50 corresponding to an implant having predetermined dimension, such as, for example, one of the implant sizes in Table 1 above. The trial 50 is inserted into the prepared joint. After inserting the trial 50, the surgeon can determine whether the trial 50 is properly sized for the resected joint. If the selected trial 50 is the proper size, the surgeon can proceed with installing the implant. If the selected trial 50 is the wrong size, the surgeon can select a larger/smaller trial. This process can be repeated until the proper trial 50 has been identified. The surgeon can then select an implant 2, 102, 202 size corresponding to the selected trial 50.

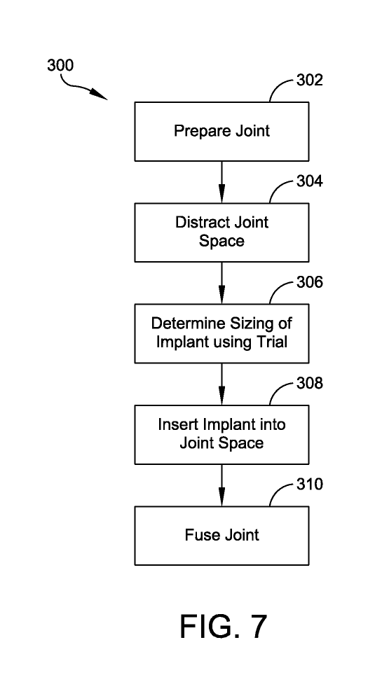

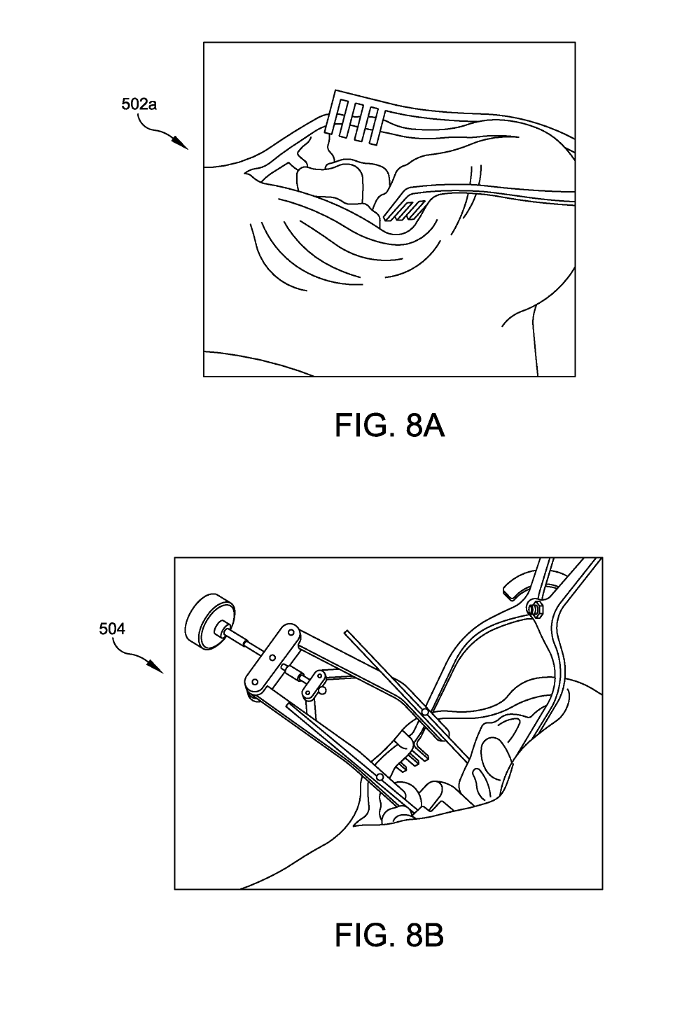

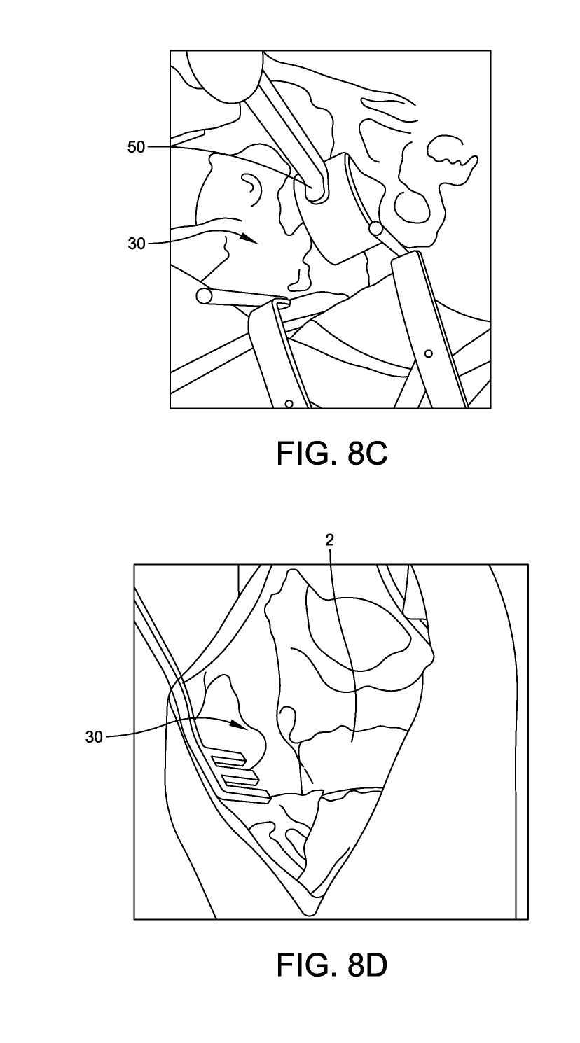

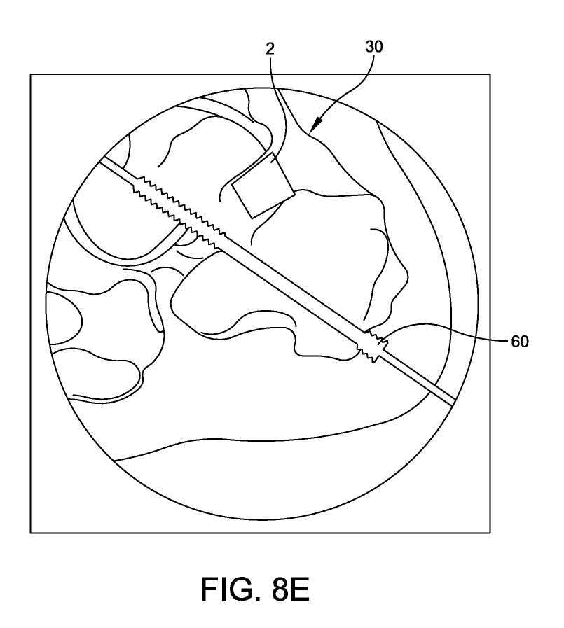

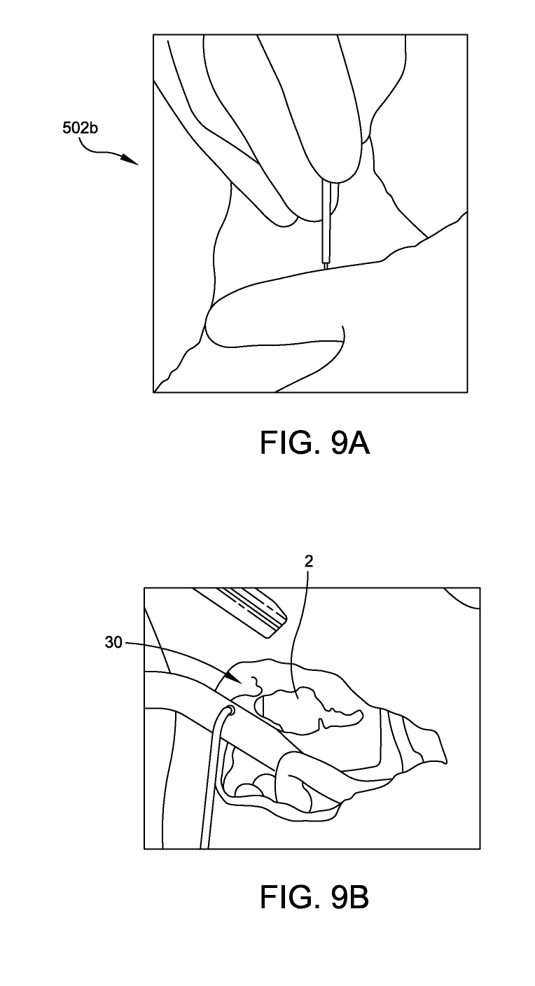

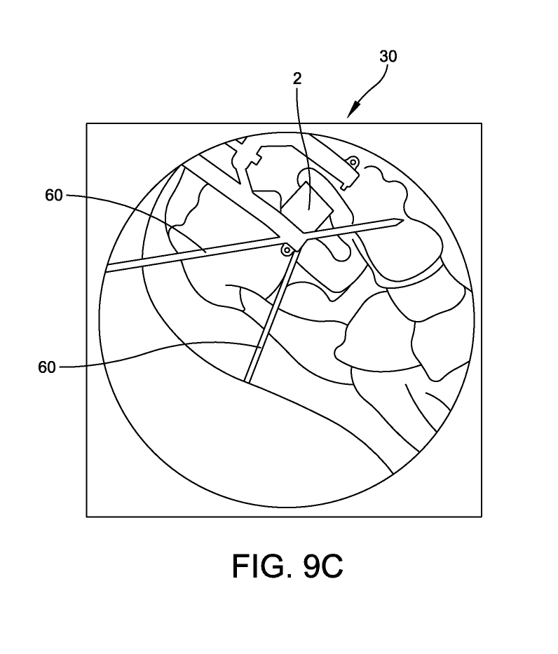

FIG. 7 is a flowchart illustrating one embodiment of a method 300 for implanting a subtalar implant. FIGS. 8A-9C illustrate various steps of the method 300. In step 302, a joint, for example the subtalar joint 30 illustrated in FIG. 2, is prepared to receive an implant 2. Preparation of the joint may comprise, for example, resecting one or more bones located at the joint, adjusting the angle between a first bone and a second bone, and/or performing any other necessary preparation of the joint. FIGS. 8A and 9A illustrate various potential joint preparation procedures. FIG. 8A illustrates one embodiment of a posterior approach 502a to a subtalar joint. FIG. 9A illustrates one embodiment of a lateral approach 502b to a subtalar joint. In each embodiment, the subtalar joint is exposed by removing covering layers of tissue. For example, in a lateral approach 502b, retraction of the peroneal tendons may be performed to expose the joint.

In step 304, the joint is distracted. The joint may be distracted using any suitable technique and/or device as known in the art. FIG. 8B illustrates one embodiment of distraction 504 of the subtalar joint. In step 306, the sizing of the implant 2 is determined using a trial, such as, for example, the trial system 40 illustrated in FIGS. 6A-6B. A trial 50 is inserted into the distracted joint. The surgeon observes the trial 50 within the joint and can select larger/smaller trials 50 until the surgeon is satisfied with the fit of the trial 50 in the joint. The size of the implant 2 corresponds to the selected trial 50. FIG. 8C illustrates one embodiment of a joint 30 having a trial 50 inserted therein. In step 308, the selected implant 2 is implanted within the distracted joint. The implant 2 is inserted such that a sidewall 6 of the implant 2 is in a load-bearing arrangement with the joint and the porous material 14 is in contact with at least one of the bones of the joint. The implant 2 may be inserted using an insertion tool 50 illustrated in FIG. 5. The insertion tool 50 is inserted through an incision made near the joint to deliver the implant 2 to the prepared joint. The implant 2 is held in the joint 30 by one or more bones after the insertion tool 50 is removed. FIGS. 8D and 9B illustrate various embodiments of an implant 2 implanted in the subtalar joint 30. In step 310, the joint is fused by, for example, a screw 36 inserted through the first bone 32 of the joint 30 and into the second bone 34 of the joint 30. The screw 36 fuses the joint 30. FIGS. 8E and 9C illustrate various embodiments of fused subtalar joints 30 having an implant 2 therein. FIG. 8E illustrates one embodiment having a single screw 60 inserted from a first bone 32 to a second bone 34 of the joint 30 to fuse the joint. FIG. 9C illustrates one embodiment having multiple screws 60 inserted into the joint. In some embodiments, a screw 60 may extend into and/or through the implant 2 to anchor the implant 2 in a fixed position.

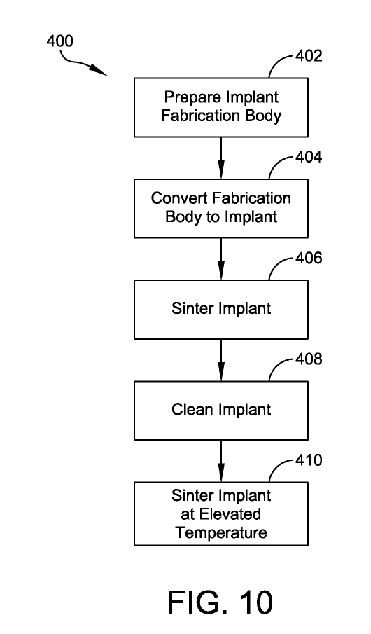

FIG. 10 is a flowchart illustrating one embodiment of a method 400 for manufacturing an implant 2 as described herein. At step 402, a fabrication body for an implant 2 is prepared. In some embodiments, the fabrication body is prepared using a suitable method, for example, using an additive manufacturing method. For example, in some embodiments, a selective laser sintering process is employed. The shape and size of the fabrication body is similar to or about the same as those of the implant 2, with consideration of possible shrinkage in later sintering processes. Computer-aided design (CAD)/Computer-aided manufacturing (CAM) technologies can be used in combination with additive manufacturing methods. The implant 2 can be designed using CAD. A model including related design parameters can be output from a computer. The related design parameters for the implant 2 as a final product include shape, configuration, dimensions, porosity, and surface roughness of each portion of the implant 2. In some embodiments, 3D imaging technology, for example, CT scan data of an actual patient, can be used with CAD/CAM technologies to assist surgeons to provide a customized model for a specific patient.

An additive manufacturing system suitable for metal generation, such as, for example, a 3D printing process or a selective laser sintering process, can be used at step 404 to convert the model into an implant based on the related design parameters. Physical parameters of the implant such as porosity and density of the material in each location can be correspondingly adjusted by the additive manufacturing system based on the model. Examples of the material used include but are not limited a metal powder such as titanium, titanium alloy or stainless steel. In some other embodiments, each portion of an implant 2 may be molded separately and then combined together to form a complete implant. The molding may be achieved through compression molding of metal powders.

At an optional step 406, at least one portion of the implant is sintered. In some embodiments, step 406 is performed using a laser during step 404 of the additive manufacturing process of the implant such that the sintering at step 406 may be performed concurrently with step 404. Laser sintering is applied while or right after each point or portion is manufactured. Direct laser sintering or selective later sintering may be used. One of ordinary skill in the art will understand that other sintering methods can be used.

At step 408, the implant is cleaned to remove excessive particles, which are not attached with or are loosely attached to the implant. Step 408 may be optional. In some embodiments, step 408 is performed before step 406 of sintering the implant at the elevated temperature. The step 408 of cleaning may be performed by applying high pressure air or other gases to the surface of the implant. The excessive particles can be blown away.

At step 410, the implant is sintered at an elevated temperature to provide the implant 2 described above. Such a sintering can be performed in an oven or furnace. The heat sintering can be performed at any suitable temperature. The heat sintering of titanium may be performed at a temperature, for example, in the range from about 1000 to about 1500° C. The temperature and time can be selected to control the physical parameters of final implant.

Although the subject matter has been described in terms of exemplary embodiments, it is not limited thereto. Rather, the appended claims should be construed broadly, to include other variants and embodiments, which may be made by those skilled in the art.

1. An implant, comprising:
a body having a sidewall defining an outer perimeter of the body, wherein the sidewall defines an inner volume; and
a porous material disposed within the inner volume, the porous material having a porosity configured to promote bone ingrowth.

2. The implant of claim 1, wherein the sidewall of the body is a solid structure configured to support a load at least equal to a maximum load of a joint.

3. The implant of claim 2, wherein the sidewall comprises a smooth surface configured to prevent bone ingrowth.

4. The implant of claim 1, wherein the sidewall comprises a metal.

5. The implant of claim 4, wherein the metal includes titanium.

6. The implant of claim 1, wherein the porosity of the porous material is about 30% to about 70% by volume.

7. The implant of claim 6, wherein the porous material includes a metal mesh.

8. The implant of claim 7, wherein the metal mesh includes titanium.

9. The implant of claim 7, wherein the porosity of the porous material is about 55% to about 65% by volume.

10. The implant of claim 1, wherein the outer perimeter of the body comprises a horse-shoe shape.

11. The implant of claim 1, wherein the outer perimeter of the body comprises a full-oval shape.

12. An implant system, comprising:
an implant comprising:
a body having a solid sidewall defining an outer perimeter of the body, wherein the solid sidewall defines an inner volume; and
a porous metal material disposed within the inner volume, the porous metal material having a porosity of about 30% to about 70% by volume; and
a bone screw sized and configured for fusing a joint.

13. The implant system of claim 12, wherein the sidewall of the body is a solid structure configured to support a load at least equal to a maximum load of a joint.

14. The subtalar implant system of claim 13, wherein the sidewall is configured to support a load at least equal to a maximum load of a subtalar joint.

15. The subtalar implant system of claim 12, wherein the sidewall includes a metal.

16. The subtalar implant system of claim 12, wherein the sidewall includes titanium and wherein the porous metal material includes titanium.

17. The subtalar implant system of claim 12, wherein the outer perimeter of the body defines a horse-shoe shape.

18. The subtalar implant system of claim 12, comprising an implantation tool.

19. A method of correcting a subtalar joint deformity, comprising:
preparing a joint for receiving an implant including a body having a sidewall defining an outer perimeter of the body, wherein the sidewall defines an inner volume having a porous material disposed therein, wherein the porous material has a porosity configured to promote bone ingrowth;
positioning the implant in the prepared joint; and
driving a screw through a first bone of the joint into a second bone of the joint to fuse the first and second bones of the joint.

20. The method of claim 18, comprising determining a size of the implant using a trial prior to positioning the implant in the prepared joint.
